(19) United States
(12) Patent Application Publication
(10) Pub. No.: US 2024/0325146 A1
(43) Pub. Date: Oct. 3, 2024

(54) Positional Markers for Medical Device

(71) Applicant: Cephea Valve Technologies, Inc., Abbott Park, IL (US)

(72) Inventors: Randolf Von Oepen, Aptos, CA (US); Prem Midha, St. Paul, MN (US); Russell Shelton, San Jose, CA (US); Cathy Bergin, Hugo, MN (US)

(73) Assignee: Cephea Valve Technologies, Inc., Abbott Park, IL (US)

(21) Appl. No.: 18/396,979

(22) Filed: Dec. 27, 2023

Related U.S. Application Data
(60) Provisional application No. 63/493,200, filed on Mar. 30, 2023.

(51) Int. Cl. A61F 2/24

(52) U.S. Cl. CPC A61F 2/2436; A61F 2250/0096

(57) ABSTRACT

The present disclosure describes devices, systems, and methods for intravascularly delivering an intravascular device to a targeted cardiac valve. In one aspect, a medical device includes a body and a plurality of positional markers positioned on the body, the plurality of positional markers being defined in part by at least one cutout and having at least one parameter selected such that the plurality of positional markers vibrates, in response to an ultrasound signal, at a resonant frequency.

### CROSS-REFERENCE TO RELATED APPLICATIONS

The present application claims priority to U.S. Provisional Ser. No. 63/493,200, filed Mar. 30, 2023, the disclosure of which is hereby incorporated by reference in its entirety as if fully set forth herein.

### BACKGROUND OF THE DISCLOSURE

Intravascular medical procedures allow therapeutic treatments in various locations within a patient's body while only requiring relatively small access incisions. An intravascular procedure may, for example, eliminate the need for open-heart surgery, reducing risks, costs, and time associated with an open-heart procedure. The intravascular procedure also enables faster recovery times with lower associated costs and risks of complications. An example of an intravascular procedure that significantly reduces procedure and recovery time and cost over conventional open surgery is a heart valve replacement or repair procedure in which an artificial valve or valve repair device is guided to the heart through the patient's vasculature. For example, a catheter is inserted into the patient's vasculature and directed to the inferior vena cava. The catheter is then guided through the inferior vena cava toward the heart by applying force longitudinally to the catheter. Upon entering the heart from the inferior vena cava, the catheter enters the right atrium. For a mitral valve replacement, the catheter may be guided across the atrial septum (e.g., via a guidewire that has already been passed through the atrial septum) into the left atrium. The distal end of the catheter may be deflected by one or more deflecting mechanisms in order to align the distal end of the catheter, as well as a medical device positioned therein, with the mitral valve. Catheter deflection can be achieved by tension cables, or other mechanisms positioned inside the catheter. Precise control of the distal end of the catheter allows for more reliable and faster positioning of a medical device and/or implant and other improvements in the procedures.

An intravascularly delivered device should be placed precisely to ensure a correct positioning of the medical device, which is important for its functionality, as the device may be difficult or impossible to reposition after the device is fully deployed from the delivery system.

### BRIEF SUMMARY OF THE DISCLOSURE

The present disclosure describes devices, systems, and methods for intravascularly delivering an intravascular (“IV”) device to a targeted cardiac valve. However, it should be understood that the concepts described herein may be applicable to various other procedures requiring (i) a medical device to enter the body for a treatment procedure, and/or (ii) a medical device to be implanted within the body.

One aspect of the disclosure provides a medical device, comprising: a body; a plurality of positional markers positioned on the body, the plurality of positional markers being defined in part by at least one cutout and having at least one parameter selected such that the plurality of positional markers vibrates, in response to an ultrasound signal, at a resonant frequency.

In one example, the medical device is a valve cover of a delivery device for a prosthetic heart valve.

In one example, the body includes: a first plurality of ribs extending from a central region of the body; and a second plurality of ribs extending, oppositely relative to the first plurality of ribs, from the central region of the body.

In one example, the medical device further defines a first plurality of slots, each of the first plurality of slots being positioned at least partially between an adjacent pair of the first plurality of ribs, and a second plurality of slots, each of the second plurality of slots being positioned at least partially between an adjacent pair of the second plurality of ribs.

In one example, the first plurality of slots is substantially T-shaped, defining a first crossbar; and the second plurality of slots is substantially T-shaped, defining a second crossbar that is distinct from the first crossbar.

In one example, the plurality of markers is positioned on at least one of the first plurality of ribs or the second plurality of ribs.

In one example, the plurality of markers is defined by the at least one cutout in at least one of the first plurality of ribs or the second plurality of ribs.

In one example, the at least one cutout extends in a direction that is parallel to a longitudinal direction of the medical device.

In one example, the plurality of markers has at least one resonant frequency such that the plurality of markers vibrates at the resonant frequency in response to an ultrasound imaging procedure.

In one example, the resonant frequency is in the range of 2-8 MHz.

In one example, the at least one parameter comprises one or more of: a material of the medical device; or a beam length associated with the plurality of markers.

Another aspect of the disclosure provides a system, comprising: the medical device; an ultrasound imaging apparatus; and a probe.

In one example, the medical device is a valve cover configured to deliver a prosthetic heart valve, wherein the plurality of markers is positioned on the valve cover in a manner that approximately aligns the prosthetic heart valve with native annulus tissue when the valve cover is in a final desired position prior to deployment of the prosthetic heart valve. In one example, the system excludes a fluoroscopy imaging apparatus.

In one example, the plurality of positional markers is configured to vibrate in response to the ultrasound signal, at the resonant frequency, when the medical device is at any angular orientation relative to the probe.

In one example, the plurality of positional markers is positioned on the body such that a position and/or orientation of the medical device relative to patient anatomy is known from a position and/or orientation of the plurality of positional markers when subjected to the ultrasound signal.

Another aspect of the disclosure provides a method of imaging a medical device within a patient's body, comprising: emitting an ultrasound signal from a probe toward the medical device while the medical device is within the patient's body; as a result of emitting the ultrasound signal, causing echogenic markers on the medical device to vibrate at a natural frequency; detecting the vibrations of the medical device; displaying a representation of the medical device on a display device based on the detected vibrations.

In one example, displaying the representation includes displaying an artifact corresponding to a position of the echogenic markers.

In one example, the medical device and anatomy of the patient are simultaneously imaged using ultrasound, without using fluoroscopic imaging.

In one example, the medical device and anatomy of the patient are simultaneously imaged using ultrasound, without using a second imaging modality in addition to ultrasound.

### BRIEF DESCRIPTION OF THE DRAWINGS

FIG. 1 illustrates a delivery system configured for delivering, positioning, and deploying an IV device, the delivery system including a handle assembly coupled to a delivery member;

FIG. 2A illustrates a transverse cross-section of the delivery member showing various delivery member components that may be utilized;

FIG. 2B illustrates a longitudinal cross-section of the delivery member;

FIG. 3 illustrates an exemplary approach for delivering an IV device to the mitral annulus;

FIG. 4 illustrates a distal end portion of the outer sheath, showing various sections that may be formed in the outer sheath;

FIG. 5 is a longitudinal cross-section of the outer sheath of FIG. 4;

FIG. 6 is a partial cut-away view of an intermediate portion of the outer sheath;

FIG. 7A depicts a side view of an exemplary valve cover according to one or more aspects of the disclosure;

FIG. 7B depicts a perspective view of a section of the valve cover of FIG. 7A;

FIG. 7C is a perspective view of a section of the valve cover of FIGS. 7A-B;

FIG. 8 is a table depicting exemplary materials and exemplary parameter values of a valve cover and the corresponding natural frequency or frequencies associated therewith;

FIG. 9A is an ultrasound image taken during ultrasound imaging of an exemplary valve cover;

FIG. 9B is the ultrasound image of FIG. 9A with an annotation overlaying an exemplary valve cover and positional markers, demonstrating the correspondence between markers and artifact;

FIG. 10 is a partial view of a valve cover according to one or more aspects of the disclosure;

FIG. 11 is a partial view of a valve cover according to one or more aspects of the disclosure;

FIG. 12 is a schematic view of a medical device incorporating positional markers according to one or more aspects of the disclosure;

FIG. 13A is a partial view of a valve cover depicting a slot;

FIG. 13B is a schematic view of the slot of FIG. 13A according to one or more aspects of the disclosure;

FIG. 13C is a schematic view of a slot of a valve cover according to one or more aspects of the disclosure;

FIG. 14A is a representation depicting the von Mises stress distribution in a valve cover bent at 90 degrees;

FIG. 14B is a representation depicting the von Mises stress distribution within the valve cover of FIG. 14A;

FIG. 15A is a representation depicting the von Mises stress within a rib during rib deflection;

FIG. 15B is a partial view of the representation of FIG. 15A; and

FIG. 16 is a representation depicting the von Mises stress distribution within a rib during application of force to deflect the rib.

### DETAILED DESCRIPTION

As used herein, the term “proximal” when used in connection with a delivery system refers to the end of the delivery system closer to the user of the delivery system when being used in an intended manner, while the term “distal” refers to the end of the delivery system farther away from the user.

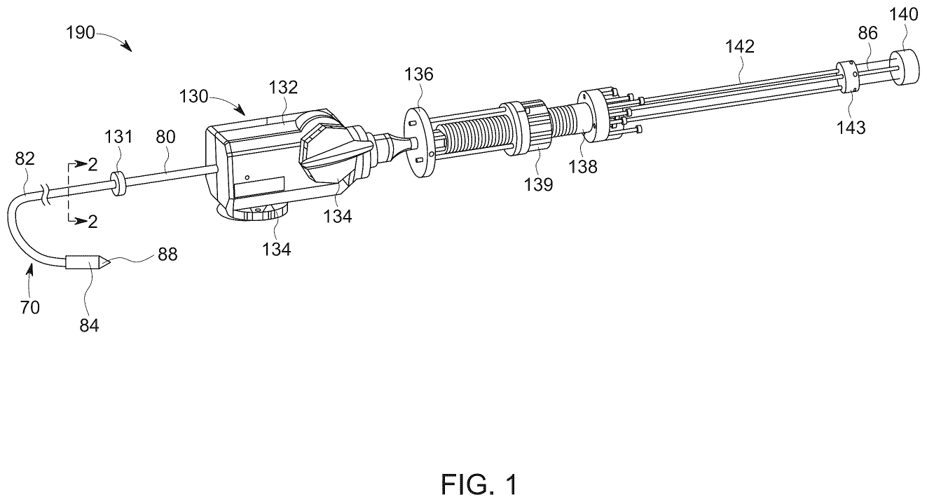

FIG. 1 illustrates an exemplary embodiment of a delivery system 190. As shown, the delivery system 190 includes a handle assembly 130 and a delivery member 70. The delivery member 70 is operably coupled to the handle assembly 130 and extends distally from the handle assembly 130. The delivery member 70 includes a plurality of catheter and/or hypotube members which provide different functionality during operation of the delivery system 190 to enable effective delivery and deployment of an intravascular (“IV”) device.

The proximal end of an outer sheath 82 is coupled to an end ring 131, and the outer sheath 82 extends to a distal tip 88. A steering catheter handle 132 is disposed proximal of the end ring 131. The proximal end of a steering catheter 80 is coupled to the steering catheter handle 132, and the steering catheter 80 extends distally from the steering catheter handle 132 into the outer sheath 82. The steering catheter handle 132 includes one or more controls 134 which are operably coupled to the steering catheter so that manipulation of the controls 134 adjusts the curvature of the steering catheter 80. For example, during delivery and deployment of a prosthetic mitral valve to a native mitral valve using a transseptal delivery route, the distal end of the delivery member 70 typically needs to be steered in at least two directions after clearing the atrial septum in order for the delivery member 70 (and the prosthetic mitral valve housed therein) to properly align with the native mitral valve.

The outer sheath 82 extends to a distal end where it is coupled to a distal piece 84 (which may also be referred to herein as a “valve cover 84”). The distal piece 84 functions to house an IV device in a compressed, pre-deployed state during intravascular delivery of the device to the targeted cardiac site.

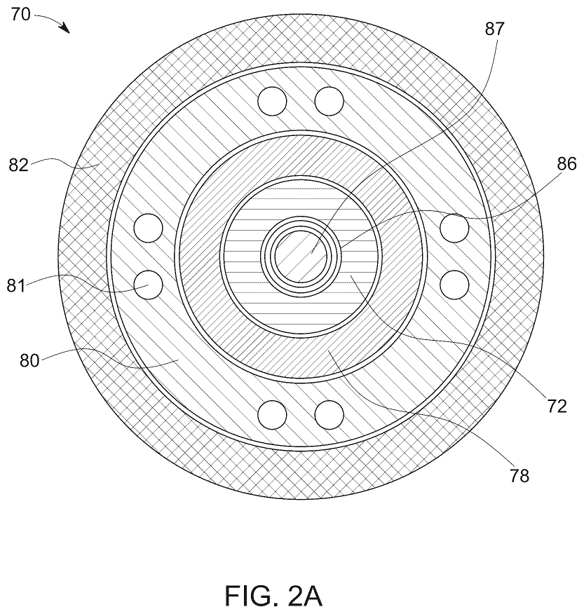

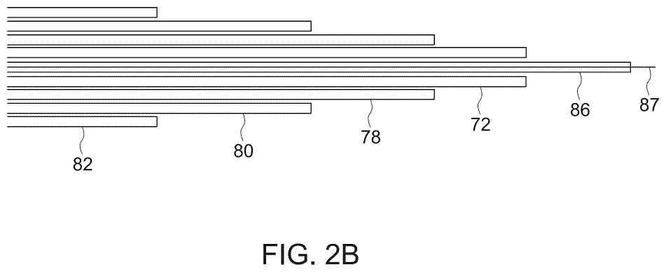

Because the steering catheter 80 is nested within the outer sheath 82, curving of the steering catheter 80 causes corresponding curving/steering in the outer sheath 82. The steering catheter 80 and outer sheath 82 may be referred to singly or collectively herein as the “outer member.” The illustrated embodiment of the delivery member 70 includes additional components which are not visible in the view of FIG. 1 but may be seen in the cross-sectional views of FIGS. 2A-B.

FIG. 2A illustrates a transverse cross-section of the delivery member 70 taken along the cross-section line 2-2 of FIG. 1. FIG. 2B illustrates a longitudinal cross-section of the delivery member 70. As shown, the steering catheter 80 is disposed radially within the outer sheath 82. An extension catheter 78 is disposed radially within the steering catheter 80. An inner catheter 72 (also referred to herein as a suture catheter) may be disposed radially within the extension catheter 78, and a guidewire tube 86 (also referred to herein as a nosecone catheter) may be disposed radially within the inner catheter 72. The guidewire tube 86 is configured for receiving a guidewire 87, and may also be configured to reversibly couple to a distal tip 88 (also referred to herein as a nosecone). Although the particular nested configuration shown in FIGS. 2A-B represents one preferred embodiment, alternative embodiments may include a different concentric arrangement of constituent parts and may include more, fewer, or different components than shown and described. For example, some embodiments may combine the steering catheter 80 and outer sheath 82 and/or configure the outermost member with steering functionality, some embodiments may include more than one catheter with steering functionality, etc.

The steering catheter 80 is configured to be selectively curved to facilitate intravascular navigation. In some embodiments, the steering catheter 80 provides steerability via a plurality of lumens 81 extending through the length of the wall of the steering catheter 80. The lumens 81 may be configured for receiving tension cables or pull wires which extend between the controls 134 and a steering ring at or near the distal end of the steering catheter 80. One or more tension cables may additionally or alternatively be coupled to intermediate sections of the steering catheter 80. Manipulation of the controls 134 therefore adjusts tension in the tension cables to increase or decrease curvature of the steering catheter 80 at various positions. In the particular example shown in FIG. 2A, each tension cable is in a looping configuration in which two ends of the steering cable are coupled to controls 134, with a middle or intermediate section of the steering cable looping around the steering ring at or near the distal end of the steering catheter 80. Thus, referring still to FIG. 2A, two pairs of diametrically opposed lumens 81 provide for steering in a first plane, while the other two pairs of diametrically opposed lumens 81 provide for steering in a second plane substantially orthogonal to the first plane. In other examples, any number of lumens 81 may be provided. For example, three pairs of lumens 81 can be provided to allow for three steering operations, including offset steering operations described in greater detail in U.S. Patent Application Publication No. 2023/0364387, the disclosure of which is hereby incorporated by reference herein. Although the controls 134 are shown here as knobs, alternative embodiments may additionally or alternatively include one or more buttons, sliders, ratcheting mechanisms, or other suitable controls capable of adjusting tension to provide steering. Illustrative structures that can be used as part of the steering catheter handle 132 and or steering catheter 80 are described in U.S. Pat. No. 7,736,388, which is incorporated herein by this reference.

Referring again to FIG. 1, a delivery catheter holder 136 is disposed proximal of the steering catheter handle 132. Although not visible in the view of FIG. 1, the proximal end of the extension catheter 78 is coupled to the delivery catheter holder 136. The extension catheter 78 extends distally away from the delivery catheter holder 136 and into the steering catheter 80. An inner catheter holder 138 (also referred to herein as suture catheter holder 138) is disposed proximal of the delivery catheter holder 136. The inner catheter 72 may be coupled to the inner catheter holder 138 so that translation of the inner catheter holder 138 corresponds to translation of the inner catheter 72. For example, the inner catheter 72 may be selectively locked relative to the inner catheter holder 138 through a set screw, clamp, or other selective holding mechanism. The inner catheter 72 extends distally away from the inner catheter holder 138 and into the delivery catheter 78.

An inner catheter control 139 is operatively coupled to the inner catheter holder 138. Manipulation of the inner catheter control 139 adjusts the relative positioning of the delivery catheter holder 136 and inner catheter holder 138, and thus the relative positioning of the extension catheter 78 and the inner catheter 72. In the illustrated embodiment, the inner catheter control 139 operates through threaded engagement with the inner catheter holder 138, such that rotation of the inner catheter control 139 translates the inner catheter holder 138 relative to the control 139 and therefore relative to the delivery catheter holder 136. Alternative embodiments may additionally or alternatively include one or more of a slider and rail assembly, a ratcheting mechanism, or other suitable means of linear adjustment.

The inner catheter 72 may extend proximally to and be attached to an inner catheter cap 143. A user may decouple the inner catheter 72 from the inner catheter holder 138 to allow movement of the inner catheter 72 by sliding/translating the inner catheter cap 143 along alignment rods 142. The guidewire tube 86 extends distally through the alignment cap 143 and into the inner catheter 72. The guidewire tube 86 extends to the distal end of the delivery member 70 where it is attached to a distal tip 88 or nosecone. The distal tip 88 or nosecone is preferably formed from a flexible polymer material and provides an angled, atraumatic shape which assists in passing the delivery member 70 through the vasculature without tearing or otherwise damaging the patient's tissue as the leading end of the delivery member 70 comes into contact with tissue. The distal tip 88 may also facilitate the leading end of the delivery member 70 passing through the inter-atrial septum to the mitral annulus, which is required in a typical transfemoral approach to the mitral annulus. In some embodiments, the distal tip 88 may be coupled to the nosecone catheter 86 only after the IV device is loaded into the distal piece 84.

In the illustrated embodiment, the guidewire tube 86 is coupled to a guidewire tube holder 140. By moving the guidewire tube handle, the guidewire tube 86 may be selectively translatable relative to the inner catheter cap 143 such that the guidewire tube 86 and distal tip 88 may be linearly translated relative to the inner catheter 72 and other components of the delivery member 70. The guidewire tube 86 may be selectively locked in a longitudinal position relative to the inner catheter holder 138 and/or inner catheter cap 143, such as through a set screw, clamp, or other selective fastener. For example, such a fastening structure may be associated with the inner catheter cap 143.

When unlocked, the guidewire tube 86 (and likewise the distal tip 88) may be moved relative to the inner catheter 72. The ability to retract the distal tip 88 relative to the inner catheter 72 reduces the risk that the distal tip 88 will become overextended during deployment, where it could become tangled in chordae tendineae and/or cause injury to cardiac tissue. Additionally, independent movement of the guidewire tube 86 (with the distal tip 88) also allows for closing the gap between the distal tip 88 and the valve cover 84 (sometimes also referred to as “valve capsule”) following deployment of the IV device. When the IV device has been released, the distal tip 88 is separated from the valve cover 84 by a distance, such as by about 40 mm. To avoid drawing air into the catheter, the gap between valve cover 84 and distal tip 88 is closed by drawing the distal tip 88 towards the valve cover 84, preferably in the left side of the heart, to avoid sucking air into the catheter when pulled back into the right side of the heart (where there is relatively low pressure).

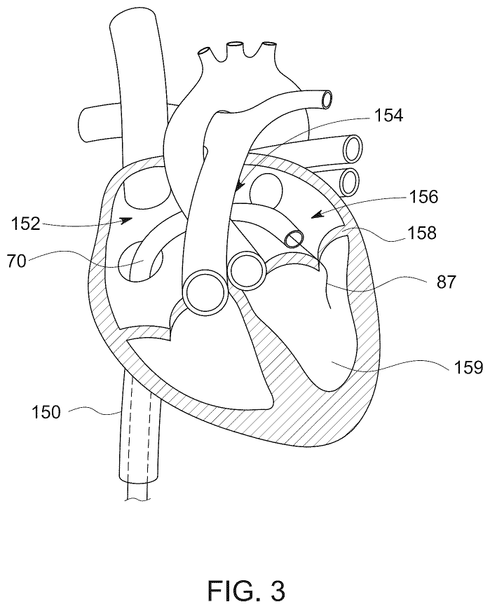

FIG. 3 illustrates a schematic representation of a patient's heart (shown in a cut-away view) and a delivery procedure to the mitral annulus that may be conducted using the illustrated delivery system 190. The delivery member 70 may be inserted into the patient's vasculature (e.g., through the femoral vein in a transfemoral approach) and directed to the inferior vena cava 150. The delivery member 70 is passed through the inferior vena cava 150 toward the heart. Upon entering the heart from the inferior vena cava 150, the delivery member 70 enters the right atrium 152. For procedures relating to the mitral valve 158, the delivery member 70 is further passed into the left atrium 156 by passing through a puncture in the inter-atrial septum 154. The puncture in the inter-atrial septum 154 may be created in a separate standard procedure, not described in more detail herein, prior to inserting the delivery system 190 into the patient. During this separate standard procedure, the guidewire 87 may be placed along the desired delivery pathway and through the atrial puncture, extending into the left atrium 156. If desired, the guidewire 87 may be further passed through the mitral valve 158 and into the left ventricle 159, as shown in FIG. 3, to provide a rail over which the delivery system 190 may ride.

In other implementations, such as for procedures associated with a tricuspid valve, the delivery member 70 may be passed through the inferior vena cava 150 and into the right atrium 152, where it may then be positioned and used to perform the procedure related to the tricuspid valve (i.e., the right atrioventricular valve). As described above, although many of the examples described herein relate to delivery to the mitral valve, one or more embodiments may be utilized in other cardiac procedures, including those involving the tricuspid valve.

Although a transfemoral approach for accessing a targeted cardiac valve is one preferred method, it will be understood that the embodiments described herein may also be utilized where alternative approaches are used. For example, embodiments described herein may be utilized in a transjugular approach, transapical approach, or other suitable approach to the targeted anatomy. For procedures related to the mitral valve or tricuspid valve, delivery of the artificial, replacement valve or other IV device is preferably carried out from an atrial aspect (i.e., with the distal end of the delivery member 70 positioned within the atrium superior to the targeted cardiac valve). The illustrated embodiments are shown from such an atrial aspect. However, it will be understood that the IV device embodiments described herein may also be delivered from a ventricular aspect.

In some embodiments, a guidewire 87 is utilized in conjunction with the delivery member 70. For example, the guidewire 87 (e.g., 0.014 in. (approximately 0.356 mm), 0.018 in. (approximately 0.457 mm), 0.035 in. (approximately 0.889 mm)) may be received within the guidewire tube 86 of the delivery member 70 as the delivery member 70 is advanced over the guidewire 87 toward the targeted cardiac valve.

Additional details regarding delivery systems and devices that may be utilized in conjunction with the components and features described herein are described in US Patent Application Publication Numbers 2018/0028177A1 and 2018/0092744A1, which are incorporated herein by this reference.

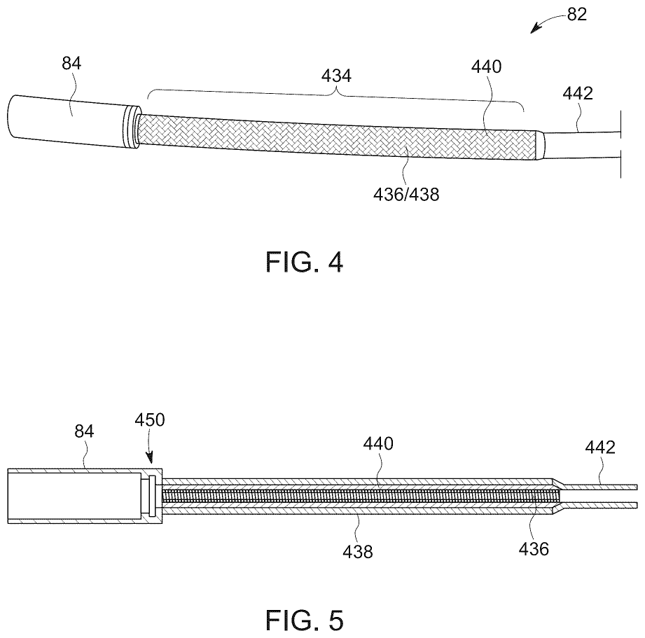

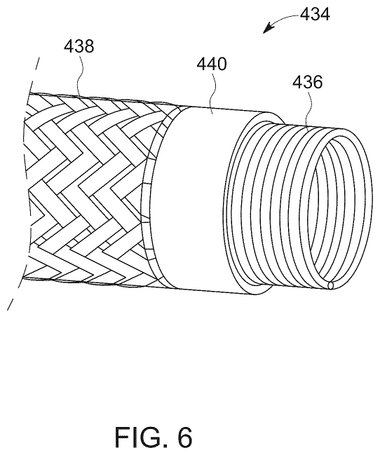

FIGS. 4 and 5 illustrate a portion of the distal end of the outer sheath 82 and distal piece 84 (also occasionally referred to herein as cover 84). Distal piece 84 can be formed as a cylindrical tube having an inner diameter and length sized to receive the IV device, in a collapsed/pre-deployed configuration, within the lumen of distal piece 84. For example, the IV device may be a collapsible and expandable prosthetic heart valve, which may include a self-expanding anchoring frame coupled to a self-expanding valve frame, with a plurality of prosthetic leaflets coupled to the valve frame to provide the valve functionality. Distal piece 84 can include a plurality of microfabricated cuts (e.g., laser cuts) and a pair of continuous longitudinal spines located on opposite sides so that distal piece 84 can bend and flex substantially in a single plane. The outer sheath 82 can also include a bendable portion 434 (sometimes referred to as “bending portion”) that can be attached to and located proximal to distal piece 84. Bendable portion 434 may have a sufficient length to surround and extend along that portion of the delivery system that is designed to bend and to be positioned, via the steerable catheter 80, to navigate through a patient's vasculature and/or heart to a target site for deploying the IV device. In the context of a transfemoral mitral valve procedure, the bendable portion 434 may generally correspond to portions of the outer sheath 82 that extend through the atrial puncture and within the right atrium during deployment of the prosthetic mitral valve. In some embodiments, the bendable portion 434 can include a cable tube or coil 436 surrounded by a braided structure 438 (sometimes collectively referred to as the “coil/braid portion 436/438”) as shown in FIG. 6.

Attached to the proximal end of bendable portion 434 is a cut hypotube 442 that extends from bendable portion 434 to the proximal end of the sheath 82. Hypotube 442 can include a plurality of slits and at least one longitudinally continuous spine that can preferably be continuous and uninterrupted along a longitudinal length of, and located at a fixed angular location on, hypotube 442.

In such embodiments, it can be desirable for the bendable portion 434 of delivery catheter to remain liquid tight. To seal the bendable portion 434, a flexible, fluid impermeable covering can be provided over the coil/braid portion 436/438, extending from the distal piece 84 to a location proximal to the coil/braid portion 436/438. For example, the delivery sheath 82 can also include a thin-walled flexible cover 440 that extends from the distal piece 84 to the hypotube 442. Flexible cover 440 can be bonded at each end to the underlying structure, using one of a variety of different adhesives, thermal adhesives, UV bonded adhesive, or other techniques.

Referring again to FIG. 5, outer sheath 82 can also be coupled to distal piece 84 via a swivel connection, generally indicated at 450. To overcome the challenging forces that can develop during insertion of a relatively large delivery catheter into the vasculature of a patient, swivel connection 450 allows rotation of outer sheath 82 by a few degrees, back and forth (i.e., alternating between clockwise rotation and counter-clockwise rotation) while at the same time moving the delivery system 190 in a generally longitudinal direction. This rotational motion (during simultaneous longitudinal translation) helps to overcome some of the longitudinal forces that may resist insertion of outer sheath 82 through a patient's vasculature or frictional forces between the outer sheath 82 and the steering catheter 80.

### Positional Markers

During delivery and/or implantation of a medical device, such as an intravascular device (e.g., prosthetic heart valve), ultrasound imaging (e.g. echocardiography) can be used in combination with fluoroscopy to visualize a position of the medical device relative to the anatomy to ensure proper placement within the body. Ultrasound imaging can use a transducer or probe (e.g., a transesophageal echocardiography or “TEE” probe) that emits an ultrasound signal, e.g., high-frequency sound waves within a known frequency band. The emitted sound pulses reflect off of the various structures in their path and are sensed by the probe from which they were emitted. These reflections can be interpreted by a signal processor of the ultrasound imaging apparatus to determine a field of view of the probe, and the field of view can be displayed by a display device to be visualized by a clinician.

In particular, during delivery and/or implantation of a prosthetic heart valve, such as a mitral valve, ultrasound imaging can be used to visualize the anatomy, such as leaflets and the annulus of a native mitral valve, while fluoroscopy can be used to visualize one or more components of the medical device which show up under x-ray. However, ultrasound imaging typically does not provide precise detail of the medical device within the patient, and fluoroscopy typically does not provide precise detail of soft tissue, including the leaflets and annulus of the native mitral valve.

As a result of the above-described limitations on ultrasound and X-ray (e.g. fluoroscopic) imaging, many intravascular procedures, including transcatheter heart valve replacements, are typically conducted with both imaging modalities performed simultaneously. Not only is it typically more complex to perform two imaging modalities simultaneously compared to only performing one mode of imaging, but X-ray imaging also involves exposure risk to both the patient and the personnel performing the medical procedure. So, while it would be desirable to be able to perform satisfactory imaging of a medical procedure using only ultrasound imaging, using both X-ray and ultrasound imaging is standard because the limitations of imaging medical devices using only ultrasound (e.g., echocardiography) have not yet been solved satisfactorily.

In certain existing systems, geometric features (e.g., dimples or divots) have been added to the medical device to increase the amount of reflected ultrasound signal to better visualize a position of the medical device during delivery and/or implantation using ultrasonic imaging. However, such systems are sensitive to the orientation of the geometric features relative to the probe and may be unreliable depending on the configuration (e.g., position and/or orientation) of device(s) relative to the anatomy during a procedure. For example, in existing systems that include divots in an attempt to provide a better return signal to the probe, the return signal is typically only enhanced when the position of the divot surface relative to the probe creates a particular angle of incidence (e.g., the angle of incidence is such that the signal reflects in the same direction of incidence). At other relative positions between the divots and the probe, the return signal may not be materially enhanced by the divots. Further, such features are costly to manufacture.

The disclosure below relates to echogenic features that enhance the ability of a medical device (or any other device temporarily or permanently positioned or implanted into the body) to be viewed with precision using ultrasound imaging by producing a robust return signal regardless of the angle of incidence. It should be understood that, although the echogenic marker features are described below in the context of one particular application of a transcatheter delivery device, the echogenic markers may be applied to any device that would benefit from accurate ultrasonic imaging, whether a delivery device that is within the body only temporarily during a particular procedure or an implantable device intended to reside within a patient temporarily or permanently.

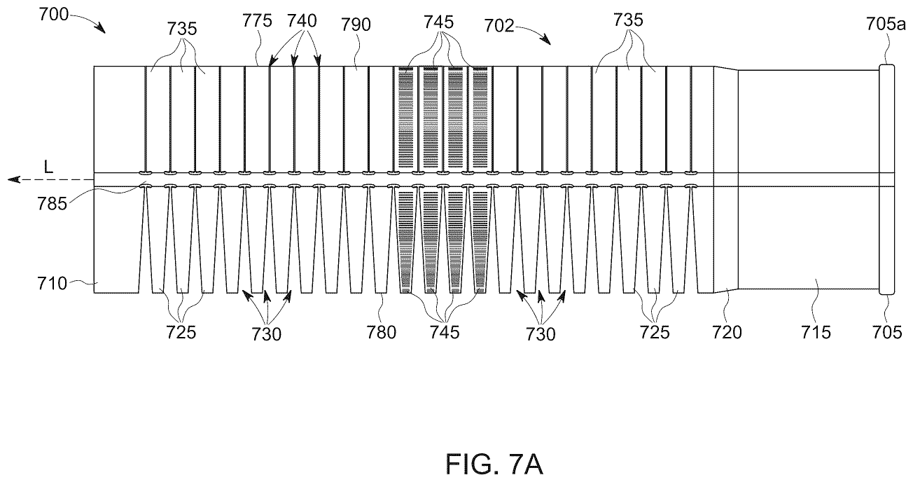

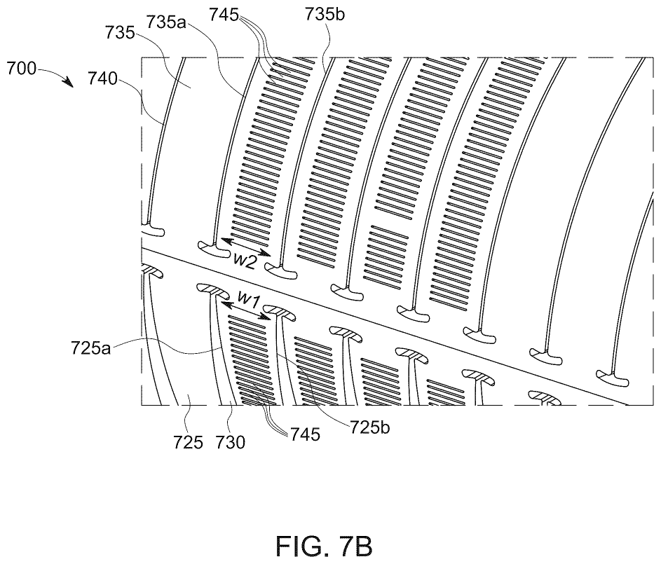

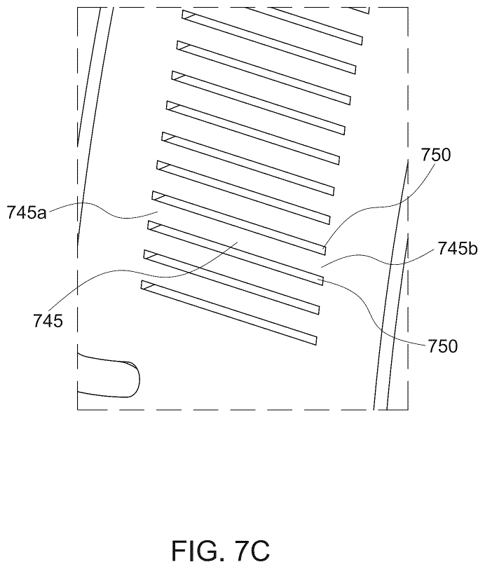

FIG. 7A depicts a side view of an exemplary valve cover 700 according to one or more aspects of the disclosure. FIG. 7B depicts a perspective view of a section of the valve cover 700 of FIG. 7A. FIG. 7C is a perspective view of a section of the valve cover 700 of FIGS. 7A-B. In one example, the valve cover 700 may be implemented in the examples described above, in particular as valve cover 84 depicted in FIG. 1.

With reference to FIG. 7A, at least a portion, or the entirety of, the valve cover 700 can be hollow and can be cylindrical or substantially cylindrical (e.g., having sections of differing diameter, rounded edges, etc.). In one example, the valve cover 700 can be a right cylinder, i.e., a cylinder having two parallel bases linked by a closed circular surface where each base is circular in shape. At least a portion, or the entirety, of the valve cover 700 can be made of metal, and in one example can be titanium, stainless steel (e.g., stainless steel 304 and/or stainless steel 316), or Nitinol. In one particular example, the valve cover can be made of Grade 23 titanium. In another particular example, the valve cover 700 can be made of a cobalt-chromium-tungsten-nickel alloy, e.g., L-605. In still another example, at least a portion, or the entirety of, the valve cover 700 can be made of plastic.

The valve cover 700 can have a body 702 including a proximal end 710 (also referred to as “trailing end”) and a distal end 705 (also referred to as “leading end”). The body 702 of the valve cover 700 can also include a connector region 715, a transition region 720, and a ribbed region 790. In the example depicted in FIG. 7A, the distal end 705 forms a lip region 705a and the connector region 715 is adjacent to and/or proximally positioned relative to the lip region 705a. The valve cover 700 can have a constant inner diameter, with the regions 705a, 715, 720, and 790 can have differing outer diameters resulting in differing wall thicknesses, as explained in greater detail below. The lip region 705a can have a greater outer diameter than the connector region 715, resulting in the connector region 715 having a reduced wall thickness as compared to one or both of the ribbed region 790 and the lip region 705a. This allows a prosthetic valve inside the valve cover 700, and in particular inside the connector region 715, to be visible under X-ray with the lip region 705a acting as a radiopaque marker. A transition region 720 is adjacent to and/or proximally positioned relative to the connector region 715, with the transition region 720 having an increasing outer diameter (in one example, linearly or constantly increasing outer diameter) and wall thickness relative to the connector region 715. The ribbed region 790 is adjacent to and/or proximally positioned relative to the transition region 720, with the ribbed region 790 having a greater outer diameter and a greater wall thickness than the connector region 715 and the transition region 720 serving as a transition of diameter and wall thickness between the connector region 720 and the ribbed region 790.

In operation, an IV device, such as a prosthetic heart valve, can be housed within the valve cover 700 in a compressed, collapsed, and/or pre-deployed state during intravascular delivery of the device to the targeted cardiac site. In this regard, the proximal end 710 can engage with an outer sheath of a delivery device (e.g., outer sheath 82 or a similar component) and the distal end 705 can engage with and/or be in contact with a nosecone (e.g., distal tip 88 or a similar component).

With reference to FIGS. 7A-B, the ribbed region 790 of valve cover 700 may include a central region 785 (also referred to as a “spine)” that generally extends along a longitudinal axis L of the valve cover 700. Although not seen in FIG. 7A, the valve cover 700 can include another central region oppositely arranged (e.g., opposite diametrically), by virtue of the cylindrical or substantially cylindrical shape, to the central region 785. Also, by virtue of the cylindrical or substantially cylindrical shape, the central region 785 can have a cross-sectional shape, in a direction transverse to the longitudinal axis L, of an arc of a circle.

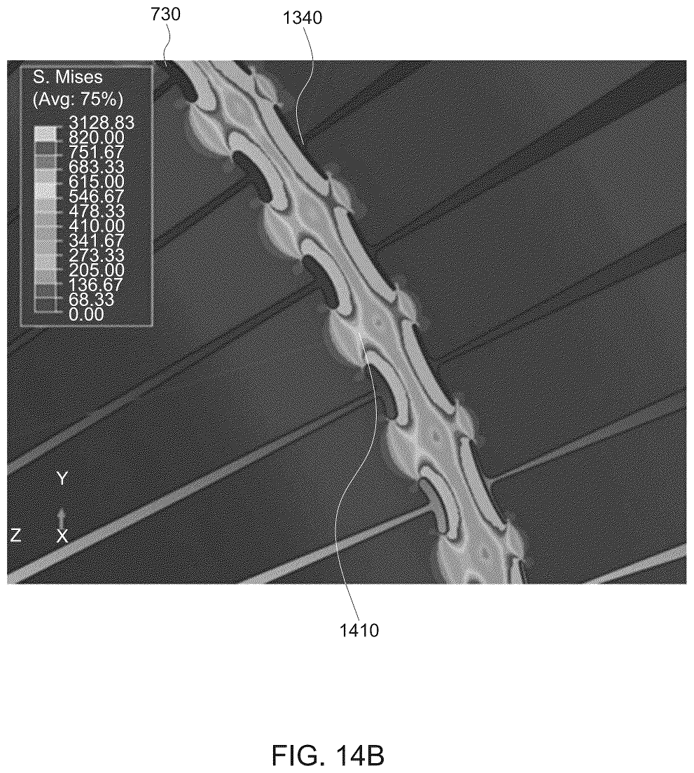

A first plurality of ribs 725 can extend between the central region 785 and the oppositely arranged central region (not shown in FIG. 7A). Between each adjacent pair of the plurality of first ribs 725 can be slots 730. As shown in FIG. 7A, at least one, or each, of the first plurality of ribs 725 can be substantially trapezoidal shaped in two-dimensional (“2D”) profile when viewed from the side (as in FIG. 7A), having a first side 725a and a second side 725b. As shown in FIG. 7B, in three-dimensional profile, at least one, or each, of the first plurality of ribs 725 can have a width w1, defined between sides 725a, b, that decreases as the rib extends further from the central region 785 to a bottom region 780 of the rib. As the rib extends from the bottom region 780 to the oppositely arranged central region (not shown), the rib can increase in width, and in one example can increase in an identical, but opposite, manner to the decreasing width described above. This configuration allows for unilateral bending, during which the slots 730 will close (as shown in FIG. 14A below). In this regard, the cross-sectional profile of the rib, in a direction transverse the longitudinal axis L, can be at least an arc of a circle and in one example can be a semicircle. The slots 730 can be T-shaped or substantially T-shaped, by virtue of the geometry of sides 725a, b, to allow for stress release when bending the valve cover 700 and as will be explained in greater detail below. In other examples, the slots 730 can take the form of a cylindrical cutout or any other suitable shape to allow for stress release.

A second plurality of ribs 735 can extend between the central region 785 and the oppositely arranged central region (not shown in FIG. 7A), and can extend oppositely from the central region 785 relative to the first plurality of ribs 725. Between each adjacent pair of the plurality of second ribs 735 can be slots 740. As shown in FIG. 7A, at least one, or each, of the second plurality of ribs 735 can be substantially rectangular shaped in two-dimensional profile when viewed from the side (as in FIG. 7A), having a first side 735a and a second side 735b. As shown in FIG. 7B, in three-dimensional profile, at least one, or each, of the second plurality of ribs 735 can have a width w2, defined between sides 735a, b, that remains constant as the rib extends further from the central region 785 to a top region 775 of the rib. As the rib extends from the top region 775 to the oppositely arranged central region (not shown), the rib can maintain the constant width, and in one example can remain constant in an identical, but opposite, manner to the constant width described above. This configuration allows for unilateral bending, during which the slots 740 will open while the slots 730 correspondingly close. In this regard, the cross-sectional profile of the rib 735, in the direction transverse to the longitudinal axis L, can be at least an arc of a circle and in one example can be a semicircle. The slots 740 can be T-shaped or substantially T-shaped, by virtue of the geometry of sides 735a, b, to allow for stress release when bending the valve cover 700 and as will be explained in greater detail below. In one particular example, the slots 740 can have a smaller width as compared to a width of the slots 730, allowing for unidirectional bending while retaining column strength against loading forces during a bending operation. In other examples, the slots 740 can be identical to the slots 730, allowing for bidirectional bending.

In use, the different shapes of the first plurality of ribs 725 compared to the second plurality of ribs 735 may provide for some bending of the valve cover 700 in a desired direction. For example, in the configuration shown in FIG. 7A, the valve cover 700 is straight and may be capable of bending or curving “downwardly” in the view of FIG. 7A, with the slots 730 closing (or decreasing in size) while the slots 740 simultaneously open (or increase in size) as the valve cover 700 bends. As the valve cover 700 returns from a bent or curved condition toward the straight condition shown in FIG. 7A, the slots 730 open while the slots 740 simultaneously close.

The valve cover 700 can also include a plurality of markers 745 that are visible (or otherwise detectable) by ultrasound imaging. As shown in FIG. 7C, the valve cover 700 can define a plurality of cutouts 750 that at least partially define the plurality of markers 745. The markers 745 can each be partially bounded by the cutouts 750 such that fixed regions 745a, b are defined. In this way, each cutout 750 is generally rectangular and each marker 745 is generally rectangular with a first axis extending between fixed regions 745a, b and a second axis perpendicular to the first axis and extending between adjacent cutouts 750. In the example of FIGS. 7A-C, the first axis is parallel to the longitudinal axis L of the valve cover 700, e.g., from proximal end to distal end.

In the example of FIG. 7A, the positional markers 745 are positioned on four of the first plurality of ribs 725 and four of the second plurality of ribs 735. In other examples, however, more or fewer of either or both of the first and second plurality of ribs 725, 735 can include the positional markers 745. Further, the positional markers 745 are preferably positioned at an anatomically (or procedurally) relevant portion of the valve cover 700. In this regard, a position and/or orientation of the valve cover 700 relative to patient anatomy during and/or after delivery and/or implantation can be known from a position and/or orientation of the positional markers 745, when subjected to ultrasound imaging, relative to the patient anatomy. For example, the positional markers 745 may be positioned on a portion of the valve cover 700 that approximately aligns with native annulus tissue at a point in the procedure where the valve cover 700 is in a final desired position prior to retracting the valve cover 700 to deploy the prosthetic heart valve maintained therein. In other examples, the positional markers 745 may be implemented at any portion or region of the valve cover 700, including other anatomically or procedurally relevant regions. In still other examples, the positional markers 745 can be positioned on a prosthetic heart valve delivered by any type of delivery system, for example the valve cover 700.

By virtue of the cutouts 750 and fixed regions 745a, b of the marker 745, each marker 745 acts as a “fixed-fixed beam” having natural frequencies corresponding to its various modes of vibration. As used herein, “natural frequencies” generally refer to the innate properties of a component (material and shape), while a “resonant frequency” is the frequency at which the component vibrates due to an external stimulus at that frequency. A component may have many natural frequencies. Such modes of vibration are governed by the following equation:

$$\\ {f}_{n} = \frac{{K}_{n}}{2 π {L}^{2}} \sqrt{\frac{EI}{ρ A}}$$

where fₙ=natural frequency, Kₙ=mode constant, F=modulus of elasticity, I=area moment of inertia, L=beam length, ρ=mass density, and A=cross-sectional area. Each mode of vibration has a mode shape and mode constant Kₙ.

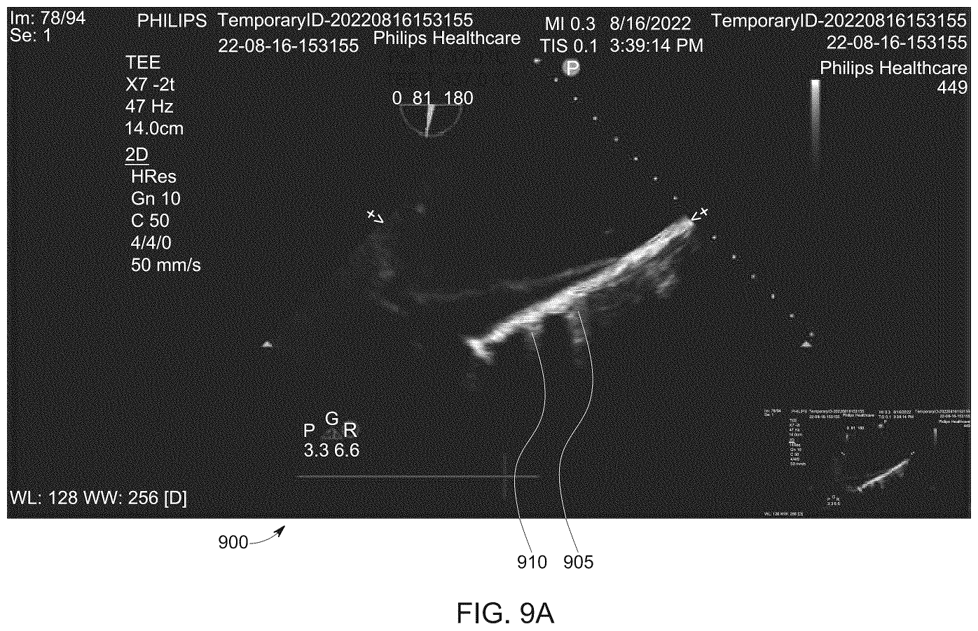

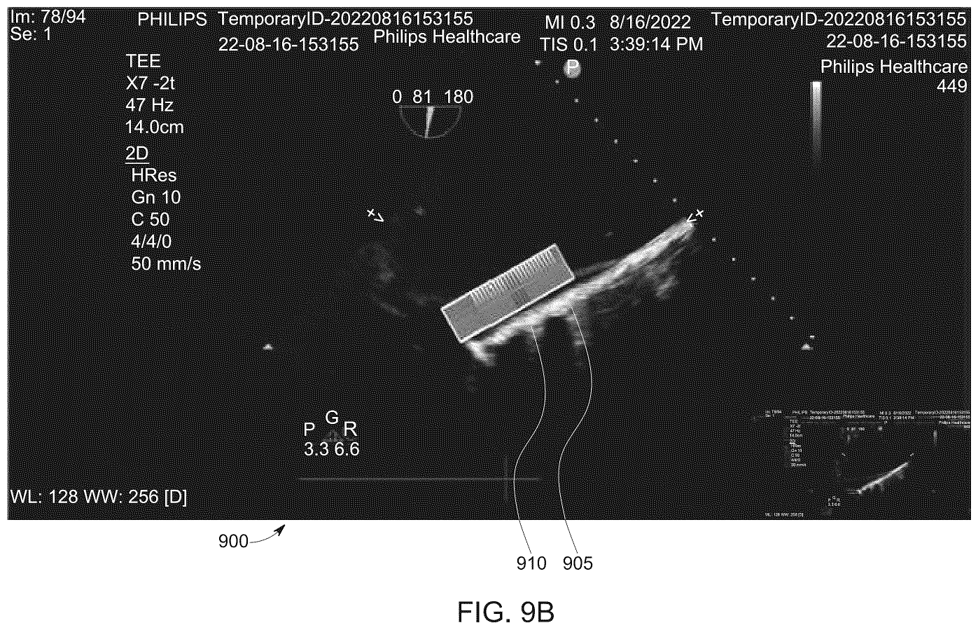

During ultrasound imaging, the ultrasound probe, which may be placed on the patient's skin, within the patient's esophagus, or in any other suitable location, is used as an external source of vibration to induce sympathetic vibrations in the markers 745 to have resonant frequencies in the range of the probe. In one example, the various parameters of the markers 745 and cutouts 750 are selected such that the markers 745 vibrate at their natural frequencies (e.g., resonant frequencies) and emit a return ultrasound signal within the 2-8 MHz band corresponding to a typical range of a TEE probe. In a further example, the parameters can be selected such that the positional markers vibrate and emit multiple harmonics within the 2-8 MHz band. This allows for better visualization and an increased likelihood of cooperation with multiple different off-the-shelf systems of varying frequency bands. In other examples, the parameters can be selected such that the positional markers vibrate and emit return signals at or near the 1-5 MHz band for transthoracic ultrasound imaging. As a result, the position of the valve cover 700 is visible and known during a procedure using ultrasound imaging, as depicted in FIGS. 9A-B.

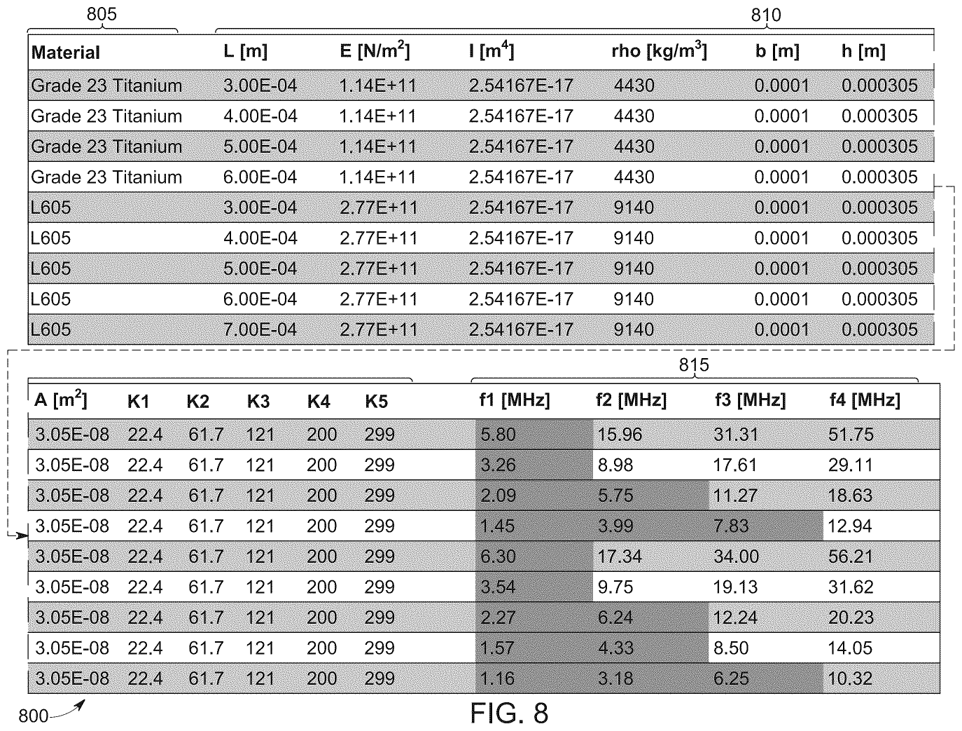

FIG. 8 is a chart 800 depicting exemplary materials 805 and exemplary parameter values 810 of a valve cover and the corresponding natural (e.g., resonant) frequency or frequencies 815 associated therewith. For example, the materials 805 can include Grade 23 titanium or Cobalt L-605. As shown, the Grade 23 titanium can have a modulus of elasticity (E) of about 1.14 E+11 N/m² and a mass density (ρ) of about 4430 kg/m³. The Cobalt L-605 can have a modulus of elasticity (L), greater than that of the Grade 23 titanium, of about 2.77 E+11 N/m² and a mass density (ρ), greater than that of the Grade 23 titanium, of about 9140 kg/m³. In the examples depicted in FIG. 8, the beam length of the beam length L can adjusted, for example by adjusting the geometry of markers 745 and/or cutouts, and can be in the range of 3.00 E-04 to 7.00 E-04 m. The resulting natural (e.g., resonant) frequency or frequencies 815 that fall within the 2-8 MHz range are highlighted. As shown, some of the material and parameter combinations yield multiple natural (e.g., resonant) frequencies 815 (e.g., harmonics) within the 2-8 MHz range. As should be understood, parameters 810 may be modified to achieve desired natural frequencies in other desired frequency range or ranges.

FIG. 9A is an ultrasound image 900 taken during ultrasound imaging of an exemplary valve cover 905 depicting an artifact 910 corresponding to the plurality of markers. FIG. 9B is the ultrasound image 900 of FIG. 9A with an annotation overlaying an exemplary valve cover and positional markers, demonstrating the correspondence between markers and artifact. In other words, during an exemplary procedure using valve cover 905, the personnel conducting the procedure may image the patient using ultrasound (e.g., via a TEE probe), and the position of the valve cover 905 within and relative to the patient's anatomy can be determined by viewing the ultrasound image 900 and determining where the artifact 910 is positioned relative to the anatomy. This artifact 910 may serve as a reliable indicator of the position of the valve cover 905, and in particular the markers positioned thereon, without the need to perform any X-ray or other imaging besides ultrasound imaging. Further, because artifact 910 is a result of the TEE probe causing the markers to vibrate at a natural frequency, the particular positioning of the TEE probe relative to the valve cover 905 is not particularly important, and artifact 910 will be visible generally independently of such positioning.

In this regard, the positional markers are positioned on the valve cover at an anatomically relevant portion of the ribbed region, e.g., where the valve cover should be finally positioned before valve deployment begins. In other examples, the positional markers may be positioned at other portions or region of the valve cover. Because the patient's anatomy is visible under ultrasound, and the anatomically (or procedurally) relevant location of the markers on the device are also simultaneously visible under ultrasound, the procedure can be guided reliably with only the single imaging modality of ultrasound, which does not create the same safety concerns as X-ray imaging, although additional imaging modalities may always be used if desired.

In another example, the markers can act as a “fixed-free beam” also having a natural frequency fₙ. As shown in FIG. 10, the markers 1045 can be defined by first cutouts 1050 and at least one second cutout 1055. In this example, the first cutouts 1050 can extend generally parallel to the longitudinal axis of the valve cover 1000 while the at least one second cutout 1055 can define a portion of an arc, by virtue of the shape of the valve cover 1000, and can be generally perpendicular to the first cutouts 1050. In this regard, the marker 1045 can have a fixed end 745b (similar to the example of FIG. 7C) and a free end 1045a. Similar equations exist for fixed-free beams with a uniform load such that the parameters, e.g., material (and associated modulus of elasticity (E) and mass density (ρ) or beam length, can be tuned to induce vibration of the positional markers within the 2-8 MHz band.

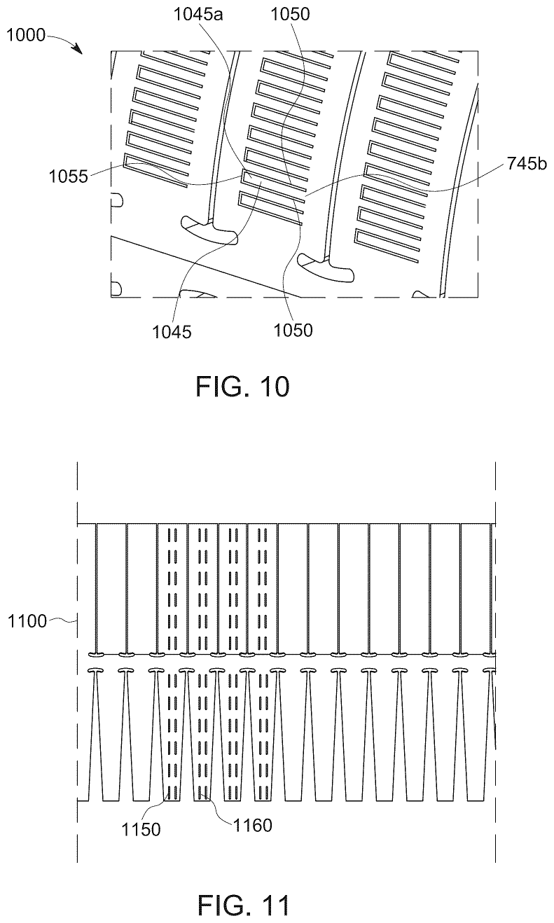

FIG. 11 depicts another exemplary valve cover 1100. In the example of FIG. 7 above, the cutouts 750 generally extended parallel to the longitudinal axis L of the valve cover. In this example, the cutouts 1150 can define a portion of an arc, by virtue of the shape of valve cover 1100, and can extend generally perpendicular to the longitudinal axis. Thus, each marker 1160 can also define a portion of an arc, by virtue of the shape of valve cover 1100, and can have a longitudinal axis also extending generally perpendicular to the longitudinal axis L of the valve cover.

FIG. 12 depicts a device 1200 (e.g., medical device) having positional markers 1210. The device 1200 can be any type of medical device and in one example can be a medical device capable of being delivered and/or implanted within a mammal (e.g., human), such as prosthetic heart valves, implantable cardioverter defibrillators (ICDs), artificial joints (e.g., hip or knee), pacemakers, hardware such as screws, pins, rods, or plates, intra-uterine devices (IUDs), or stents.

Advantageously, implementation of the positional markers on a medical device allows for precise detail to be resolved regarding the position and/or orientation of the medical device with respect to the surrounding anatomy. In particular, in the case of implantation and/or delivery of a prosthetic heart valve, a single imaging method (e.g., ultrasound imaging and free of (without) fluoroscopy) can be used to both resolve a delivery system for a prosthetic heart valve as well as the surrounding anatomy (e.g., leaflets and annulus of native heart valve). In another example, where the positional markers are on a prosthetic heart valve itself, the position of the prosthetic heart valve, as well as the surrounding anatomy (e.g., leaflets and annulus of native heart valve) can be resolved with a single imaging method (e.g., ultrasound imaging and free of (without) fluoroscopy). While the positional markers can advantageously be used with a single imaging modality as described above, in other examples the positional markers can be used with other imaging modalities, including radiopaque markers under x-ray fluoroscopy, or any other known imaging techniques.

Further advantageously, use of the positional markers is not sensitive to the orientation and/or position of the medical device relative to the ultrasound probe. Stated another way, if the ultrasound emitted by the probe reaches the positional markers, then a return signal will be generated and sensed by the probe for any orientation and/or position of the medical device relative to patient anatomy and/or probe and thus visualization can occur. Further, the use of laser cut positional markers is cost effective compared to the approaches of certain existing systems, particularly compared to the creation of dimples or divots described above.

### Slot Configuration of Valve Cover

During delivery and/or deployment (e.g., sheathing or unsheathing) of a prosthetic heart valve, the valve cover 700 is configured to bend in a steering operation during delivery of the device. During the bending, the proximal end 710 and at least a portion, or the entirety of, the ribbed region 790 are configured to bend away from the longitudinal axis L. As explained above, the slots 730 may close (or decrease in size) while the slots 740 simultaneously open (or increase in size) as the valve cover 700 bends from the straight condition. As the valve cover 700 returns from a bent or curved condition toward the straight condition shown in FIG. 7A, the slots 730 open while the slots 740 simultaneously close. In certain circumstances, the stresses caused by bending, sheathing or unsheathing the prosthetic heart valve may result in yielding of one or more ribs and may result in breaking of one or more ribs.

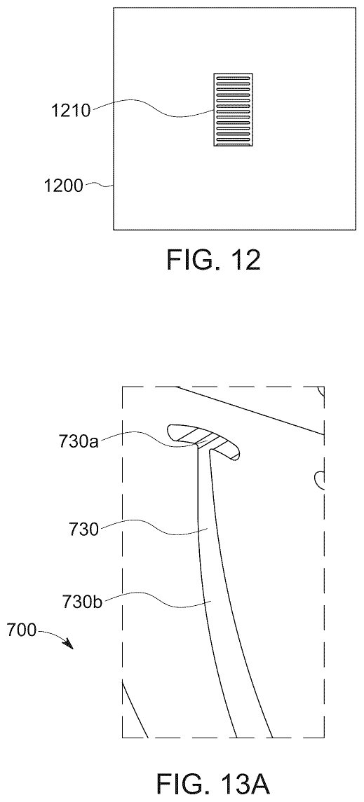

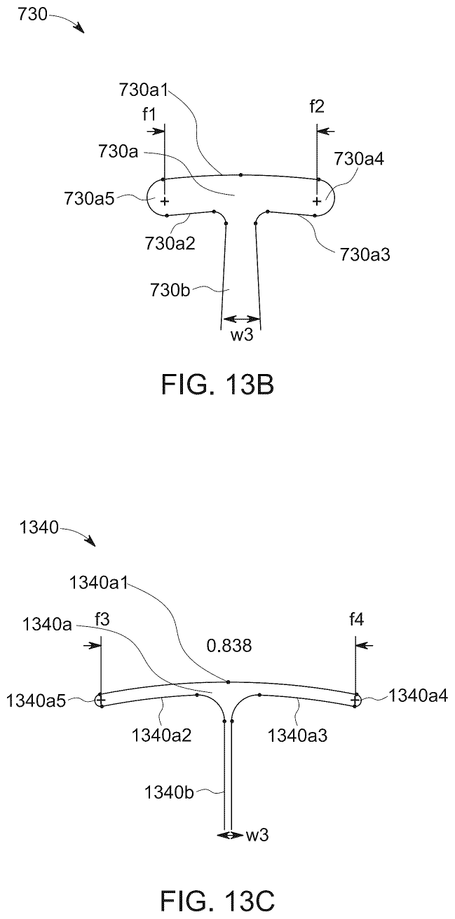

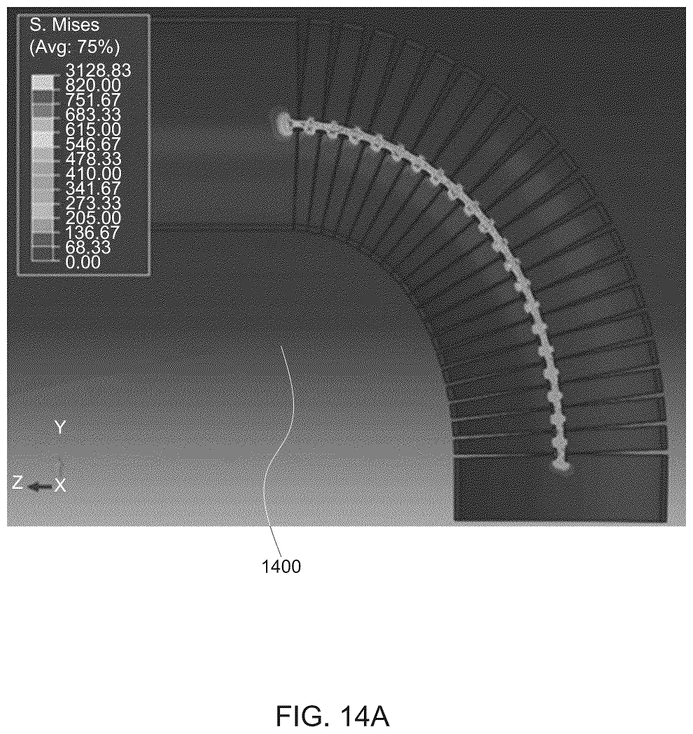

FIG. 13A is a partial view of a valve cover depicting a slot, and FIG. 13B is a schematic view of the slot of FIG. 13A according to one or more aspects of the disclosure. As described above with respect to FIGS. 7A-C, the valve cover 700 can include one or more slots 730. The slots 730 can be T-shaped or substantially T-shaped. In this regard, the slots 730 can include a crossbar 730a and a stem 730b, both of which are voids, cutouts or recesses having shapes defined by the remaining structure of the valve cover 700. The crossbar 730a can be substantially stadium shaped. In this regard, a typical stadium shape is defined as a geometric figure having a rectangular middle section capped by semicircles at each end. The crossbar 730a different from a typical stadium shape in that the middle portion comprises arcuate sections 730a1, 730a2, and 730a3, with the end portions 730a4, 730a5 defining semicircles. The stadium shape can define foci f1 and f2, corresponding to radii of the end portions 730a4, 730a5, with a distance between the foci being approximately 0.5 mm, e.g., 0.5 mm+/−0.05 mm.

The stem 730b can define a width w3 between adjacent ribs. The width w3 can decrease as the rib extends farther from the central region 785 to a bottom region 780 of the rib.

FIG. 13C depicts a slot 1340 according to one or more aspects of the disclosure. The slots 1340 can be T-shaped or substantially T-shaped. In this regard, the slots 1340 can include a crossbar 1340a and a stem 1340b. The crossbar 1340a can be substantially stadium shaped. In this regard, a typical stadium shape is defined as a geometric figure having a rectangular middle section capped by semicircles at each end. The crossbar 1340a different from a typical stadium shape in that the middle portion comprises arcuate sections 1340a1, 1340a2, and 1340a3, with the end portions 1340a4, 1340a5 defining semicircles. The stadium shape can define foci f3 and f4, corresponding to radii of the end portions 730a4, 730a5, with a distance between the foci being approximately 0.838 mm, e.g., 0.838 mm+/−0.05 mm.

The stem 1340b can define a width w4 between adjacent ribs. The width w4 can remain constant as the rib extends farther from the central region 785 to a top region 775 of the rib.

The slot 730 can have a distance between foci f1, f2 that is smaller than a distance between foci f3, f4 of slot 1340. Further, a radius of the semicircles at end portions 730a4, 730a5 is greater than a radius of the semicircles at end portions 1340a4, 1340a5.

In one example, the first plurality of ribs 725 can incorporate the slots 730 and the second plurality of ribs 735 can incorporate slots 1340. Incorporation of the slots 730 between the first plurality of ribs 725 and the slots 1340 between the second plurality of ribs 735 can advantageously reduce yielding or breakage of ribs during a sheathing or unsheathing operation.

FIG. 14A depicts a valve cover 1400 bent at 90 degrees. FIG. 14B is a chart depicting the von Mises stress distribution within the valve cover of FIG. 14A. As shown, the valve cover incorporates the slots 730 and the slots 1340. As shown in FIG. 14B, the bending does not result in yielding of the material from one section to another, as generally indicated by the reduced stress section 1410 between adjacent slots 730.

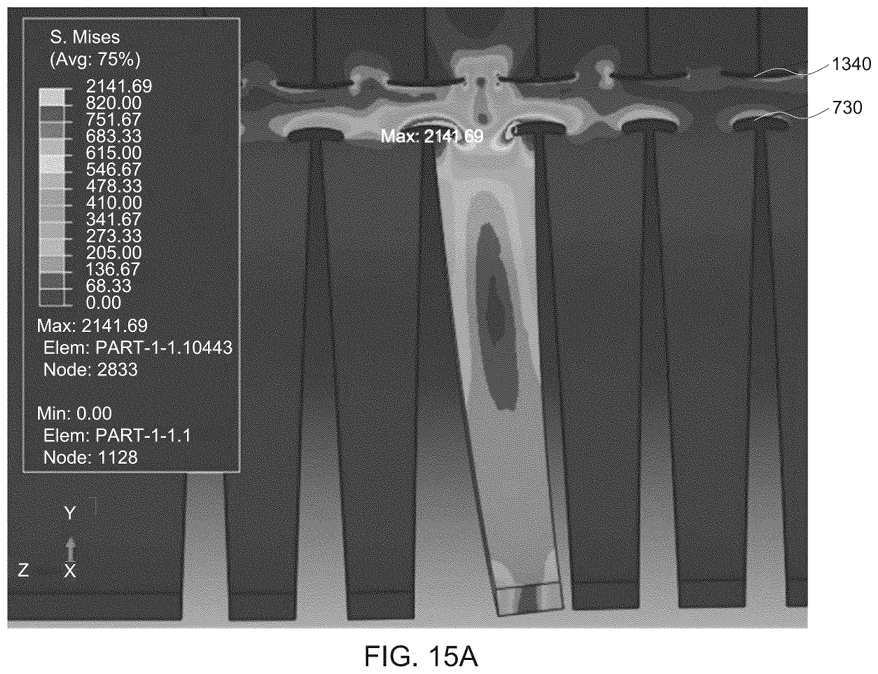

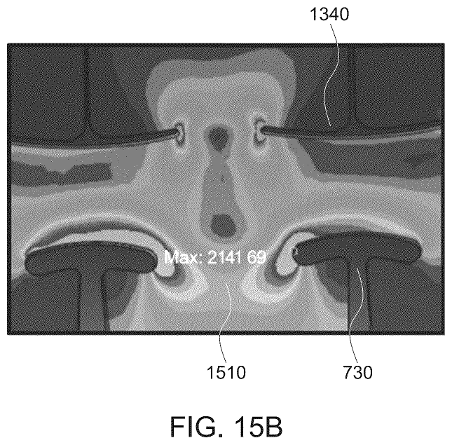

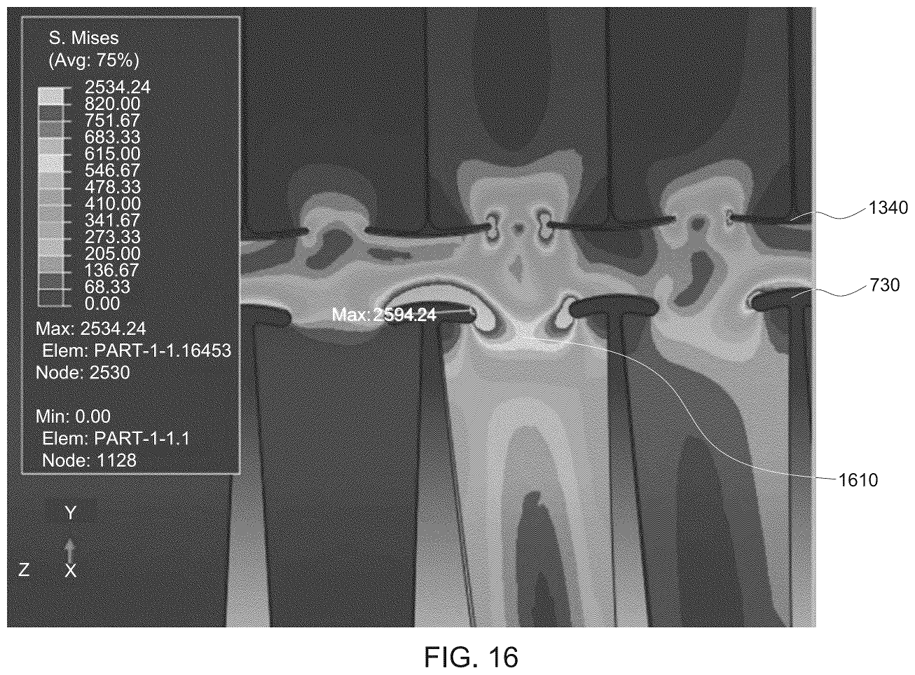

FIG. 15A is a schematic diagram and chart depicting the von Mises stress distribution within a rib during rib deflection by 0.53 mm. FIG. 15B is a partial view of the schematic diagram of FIG. 15A. As shown in FIG. 15B, the deflection of the rib does not result in yielding of the material from one section to another, as generally indicated by the reduced stress section 1510 between adjacent slots 730.

FIG. 16 is a schematic diagram and chart depicting the von Mises stress distribution within a rib during application of approximately 9 Newtons of force to deflect the rib. As shown in FIG. 16B, the force applied to the rib does not result in yielding of the material from one section to another, as generally indicated by the reduced stress section 1610 between adjacent slots 730. Reduction of stress between adjacent slots can advantageously provide a more durable valve cover that is less prone to permanent deformation and/or breakage.

### CONCLUSION

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical device, comprising:
a body;
a plurality of positional markers positioned on the body, the plurality of positional markers being defined in part by at least one cutout and having at least one parameter selected such that the plurality of positional markers vibrates, in response to an ultrasound signal, at a resonant frequency.

2. The medical device of claim 1, wherein the medical device is a valve cover of a delivery device for a prosthetic heart valve.

3. The medical device of claim 1, wherein the body includes:
a first plurality of ribs extending from a central region of the body; and
a second plurality of ribs extending, oppositely relative to the first plurality of ribs, from the central region of the body.

4. The medical device of claim 3, further defining a first plurality of slots, each of the first plurality of slots being positioned at least partially between an adjacent pair of the first plurality of ribs, and a second plurality of slots, each of the second plurality of slots being positioned at least partially between an adjacent pair of the second plurality of ribs.

5. The medical device of claim 4, wherein:
the first plurality of slots is substantially T-shaped, defining a first crossbar; and
the second plurality of slots is substantially T-shaped, defining a second crossbar that is distinct from the first crossbar.

6. The medical device of claim 3, wherein the plurality of markers is positioned on at least one of the first plurality of ribs or the second plurality of ribs.

7. The medical device of claim 6, wherein the plurality of markers is defined by the at least one cutout in at least one of the first plurality of ribs or the second plurality of ribs.

8. The medical device of claim 7, wherein the at least one cutout extends in a direction that is parallel to a longitudinal direction of the medical device.

9. The medical device of claim 1 or 7, wherein the plurality of markers has at least one resonant frequency such that the plurality of markers vibrates at the resonant frequency in response to an ultrasound imaging procedure.

10. The medical device of claim 9, wherein the resonant frequency is in the range of 2-8 MHz.

11. The medical device of claim 1, wherein the at least one parameter comprises one or more of: a material of the medical device; or a beam length associated with the plurality of markers.

12. A system, comprising:
the medical device of claim 1;
an ultrasound imaging apparatus; and
a probe.

13. The system of claim 12, wherein the probe is a transesophageal echo (TEE) probe.

14. The system of claim 13, wherein the medical device is a valve cover configured to deliver a prosthetic heart valve, wherein the plurality of markers is positioned on the valve cover in a manner that approximately aligns the prosthetic heart valve with native annulus tissue when the valve cover is in a final desired position prior to deployment of the prosthetic heart valve.

15. The system of claim 12, wherein the system excludes a fluoroscopy imaging apparatus.

16. The system of claim 12, wherein the plurality of positional markers is configured to vibrate in response to the ultrasound signal, at the resonant frequency, when the medical device is at any angular orientation relative to the probe.

17. The medical device of claim 1, wherein the plurality of positional markers is positioned on the body such that a position and/or orientation of the medical device relative to patient anatomy is known from a position and/or orientation of the plurality of positional markers when subjected to the ultrasound signal.

18. A method of imaging a medical device within a patient's body, comprising:
emitting an ultrasound signal from a probe toward the medical device while the medical device is within the patient's body;
as a result of emitting the ultrasound signal, causing echogenic markers on the medical device to vibrate at a natural frequency;
detecting the vibrations of the medical device;
displaying a representation of the medical device on a display device based on the detected vibrations.

19. The method of claim 18, wherein displaying the representation includes displaying an artifact corresponding to a position of the echogenic markers.

20. The method of claim 18, wherein the medical device and anatomy of the patient are simultaneously imaged using ultrasound, without using fluoroscopic imaging.

21. The method of claim 18, wherein the medical device and anatomy of the patient are simultaneously imaged using ultrasound, without using a second imaging modality in addition to ultrasound.
